(19) United States
(12) Patent Application Publication
(10) Pub. No.: US 2019/0314164 A1
(43) Pub. Date: Oct. 17, 2019

(54) SPINAL FUSION IMPLANT AND RELATED METHODS

(71) Applicant: Brahm Holdings, LLC, Germantown, TN (US)

(72) Inventors: Timothy R. BRAHM, Germantown, TN (US); Patrick Curlee, Germantown, TN (US)

(21) Appl. No.: 16/385,241

(22) Filed: Apr. 16, 2019

Related U.S. Application Data
(60) Provisional application No. 62/658,173, filed on Apr. 16, 2018.

(51) Int. Cl. A61F 2/44; A61F 2/46; A61L 27/36

(52) U.S. Cl. CPC A61F 2/442; A61F 2/4601; A61F 2/4611; A61F 2/4455; A61F 2002/30059; A61L 27/365; A61F 2220/0025; A61L 2430/40; A61L 27/3608

(57) ABSTRACT

A spinal graft is provided. Methods for stabilizing the spine and methods for delivering at least one flowable composition to a spinal region are also provided.

### CROSS-REFERENCE TO RELATED APPLICATIONS

The present application claims priority to U.S. Ser. No. 62/658,173 filed Apr. 16, 2018, the contents of which are incorporated in their entirety.

### FIELD OF THE INVENTION

An compressible and expandable spinal graft is provided. Methods for stabilizing the spine following such surgery are also provided.

### BACKGROUND OF THE INVENTION

Various surgeries may be performed anywhere along the spine to remove any structures that are compressing the nerves in the spinal canal or vertebral foramen (opening through which the spinal cord passes). Lamina, the bone that forms the backside of the spinal canal, may be removed along with other soft tissues to allow for more room for the nerves. The laminae are a part of the posterior arch of the vertebrae. The laminae comprise two flattened plates that extend medially from the pedicles and meet at the spinous process to form the posterior wall of the spinal foramen.

One of a variety of decompression surgeries may be performed including laminectomy, laminotomy, foraminotomy, or laminaplasty. Spinal fusion, a surgical technique used to join two or more vertebrae, is another surgical intervention option that is often performed in combination with such decompression procedures to immobilize the affected vertebrae and stabilize sections of the spine. Fusion may use a combination of bone graft(s), rods and screws to connect to vertebrae together, thereby inducing bony incorporation and healing the vertebrae together as one piece of bone. Fusion helps prevent recurrence of spinal stenosis and aids in eliminating pain arising from an unstable spine. Supplementary bone tissue (e.g., autologous iliac crest bone, allograft tissue, synthetic cage with bone substitute filler) is used in conjunction with the body's natural bone growth processes to fuse the vertebrae. Autologous tissue recovery, however, often leads to graft site morbidity. Furthermore, fusion often leads to adjacent segment disease, which may limit the duration of success of the operation. Thus, there remains a need for safe, effective grafts that are both compressible and expandable as well as methods for treating the spine after surgical intervention.

### SUMMARY OF THE INVENTION

A compressible and expandable graft is provided. The graft includes a shell having a first side and a second opposing side. The first side includes cortical bone. The second, opposing side includes compressed cancellous bone. The graft is shaped to be placed between facets and pedicals in a spine. According to one embodiment, the compressed cancellous bone may be infused with and deliver at least one flowable composition to a spine. According to one embodiment, the flowable composition includes at least one liquid component selected from the group consisting of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, PRP, and a combination thereof. According to one embodiment, the flowable composition includes a flowable birth tissue material composition. According to one embodiment, the flowable birth tissue composition comprises one or more components of the placental organ. According to one embodiment, the one or more of the components of the placental organ is selected from the group consisting of the umbilical cord, the umbilical cord blood, the chorionic membrane, the amniotic membrane, the amnion membrane, the Wharton's jelly, the amniotic fluid, and other placental gelatins, cells, and extracellular material. According to one embodiment, further includes one or more natural or recombinant bone morphogenetic proteins (BMPs). According to one embodiment, the bone morphogenetic protein is BMP-2, BMP-7 or a combination thereof. According to one embodiment, the graft further includes one or more synthetic-based bone graft extenders. According to one embodiment, the one or more synthetic-based bone graft extenders is calcium phosphate, tricalcium phosphate, phosphate, calcium sulfate, bioactive glass, or a combination thereof. According to one embodiment, the graft also includes one or more bioactive agents. According to one embodiment, the graft also includes one or more synthetic polymers.

According to one aspect, a method of stabilizing a vertebral region of a spine is provided. According to one embodiment, the method includes the step of providing a graft as provided herein. The method further includes the step of applying the graft to a vertebral region of the spine such that the second, opposing side is placed into a spinal gutter. According to one embodiment, the method further includes the step of securing the graft to the vertebral region with at least one screw, rod, plate, or a combination thereof.

According to one aspect, a method for delivering at least one flowable composition to a spinal region is provided. The method includes the steps introducing at least one flowable composition to the second, opposing side of a graft as provided herein and introducing the graft to the spinal region of a patient in need treatment. The at least one flowable composition is delivered in a time release manner. According to one embodiment, the flowable composition includes at least one liquid component selected from the group consisting of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, PRP, and a combination thereof. According to one embodiment, the flowable composition includes a flowable birth tissue material composition.

According to another aspect, a kit for stabilizing the spine is provided. According to one embodiment, the kit includes a graft as provided herein, optionally, at least one screw, rod, or combination thereof for securing the graft, and instructions for use thereof.

### BRIEF DESCRIPTION OF THE DRAWINGS

FIG. 1 is a front view of an compressible and expandable graft according to one embodiment;

FIG. 2 is a top view of the compressible and expandable graft according to the embodiment of FIG. 1;

FIG. 3 is a front view of the compressible and expandable graft according to the embodiment of FIG. 1 upon positioning in the chosen vertebral region; and

FIG. 4 is a front view of an compressible and expandable graft according to an alternative embodiment.

### DETAILED DESCRIPTION OF THE INVENTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

As used herein, “birth tissue” encompasses one or more of the components of the placental organ including, but not limited to, the umbilical cord, the umbilical cord blood, the chorionic membrane, the amniotic membrane (intact with no layers removed), the amnion, the Wharton's jelly, the amniotic fluid, and other placental gelatins, cells, and extracellular material.

As used herein, “placental tissue components” encompass one or more of the tissue components of the placental organ including, but not limited to, the umbilical cord, the umbilical cord blood, the chorionic membrane, the amniotic membrane (intact with no layers removed), the amnion, the Wharton's jelly and other placental gelatins, cells and extracellular material.

As used herein, the term “effective amount” refers to an amount of a particular composition sufficient to elicit the desired therapeutic effects.

As used herein, the term “arthrodesis surgery” refers to a procedures for linking, fusing or otherwise welding bones together. Such a surgery may function to prevent motion between vertebral bodies, repair a fracture or stabilize a spinal deformity.

As used herein, the term “decompression surgery” refers to laminectomy, laminotomy, foraminotomy, laminaplasty, or other spinal surgery where spinal fusion or stabilization interventions may be utilized.

As used herein, the term “laminectomy” refers to the surgical procedure for removing the entire lamina, a portion of the facet joints, and any thickened ligaments overlying the spinal cord and nerves.

As used herein, the term “laminotomy” refers to the surgical procedure for removing a small portion of the lamina and ligaments, usually on a single side.

As used herein, the term “foraminotomy” refers to the surgical procedure for removal of bone around the neural foramen and can be performed with a laminectomy or laminotomy.

As used herein, the term “laminaplasty” refers to the surgical procedure for the expansion of the spinal canal by cutting the laminae on one side and swinging the laminae open.

As used herein, the term “time release” refers to the delivery of a liquid component gradually over a period of time.

As used herein, the term “biologic” refers to a type of liquid component that aids in the healing cascade, aids in the spinal fusion process, or a combination thereof.

Provided herein is a spinal graft that optionally fuses to the spine upon placement. According to one embodiment, a spinal graft as provided herein stabilizes the spine. According to one embodiment, the graft is compressible and expandable and may aid in preventing spinal stenosis recurrence and eliminating pain associated with an unstable spine. The graft as provided herein is particularly useful for stabilization of the vertebrae after a spinal decompression surgery. The graft may be coated with a birth tissue composition. By providing such a coating, adhesion, nerve damage, pain, and graft migration are reduced or eliminated. Further, the incidence of graft rejection is substantially reduced thereby minimizing the potential need for additional surgery. Also provided herein are methods of stabilizing the spine after decompression surgery.

An compressible and expandable spinal graft as provided herein may be of various shapes and sizes depending on the ultimate use of the graft. According to one embodiment, the spinal graft as provided herein is of a shape and size to function as a structural support for the spine, typically in place of a disc or bone that was removed. According to one embodiment, the spinal graft as provided herein is of a shape and size to function as an onlay that includes a plurality or mass of bone fragments that grow together to stabilize the spine and bridge any joint. According to one embodiment, the spinal graft as provided herein is of a shape and size to provide a scaffold or foundation for new bone growth.

According to one embodiment, an exterior surface or shell of the spinal graft is cortical bone on first side and cancellous bone on a second, opposing side. According to one embodiment, the spinal graft as provided herein is shaped to fit between facets and pedicles in the spine. According to one embodiment, the underside of the spinal graft that is placed into the spine gutter created during surgery is compressed cancellous bone that can be infused with a flowable composition that include any liquid component such as a biologic as provided herein to help the fusion healing process. According to one embodiment, the flowable composition includes one or more vitamins, nutrients, inflammatory inhibitors, antibiotics, cytokines, minerals, growth factors, hyaluronic acids, cellular attractants, scaffolding reagents, antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, PRP, a flowable birth tissue composition, or any combination thereof.

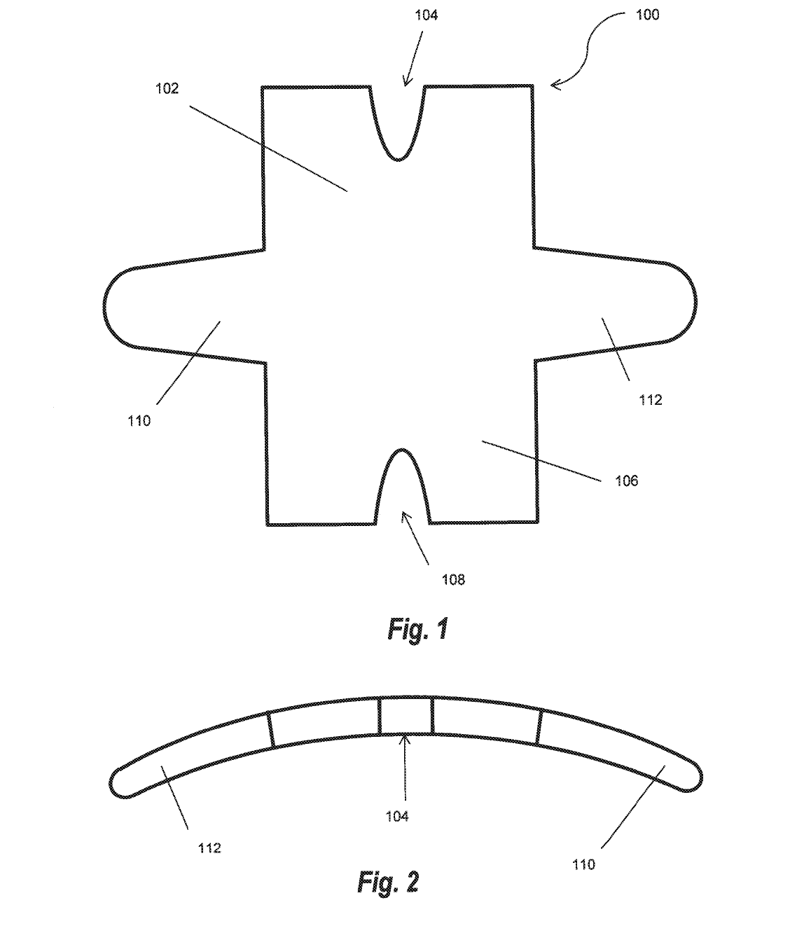

FIGS. 1 and 2 illustrate a spinal graft 100 according to a particular embodiment. As illustrated, the graft includes a specific shape that includes a superior arm 102 having a surface defining a notch 104 positioned at a central region of the superior arm 102 and an inferior arm 106 having a surface defining a notch 108 positioned ata central region of the inferior arm 106. While the arms (102, 106) are generally shown as having blunt or sharp, rectangular edges, the arms (102, 106) may also be generally rounded. The graft 100 further includes a first wing 110 and a second wing 112. While the wings (110, 112) are generally shown as having curved edges, the wings (110, 112) may also have blunt or squared edges. As illustrated in FIG. 2, the graft 100 is formed in a curved manner. By being formed in a curved manner, the graft 100 conforms to the natural shape of the spine. The graft 100 may also have varied cross-sectional shapes to conform to the varied anatomical shapes of the interspinous spaces of the spine. The graft 100 or a portion thereof may also form a barrier to keep the dural sac from connecting to or touching surrounding tissue or bone.

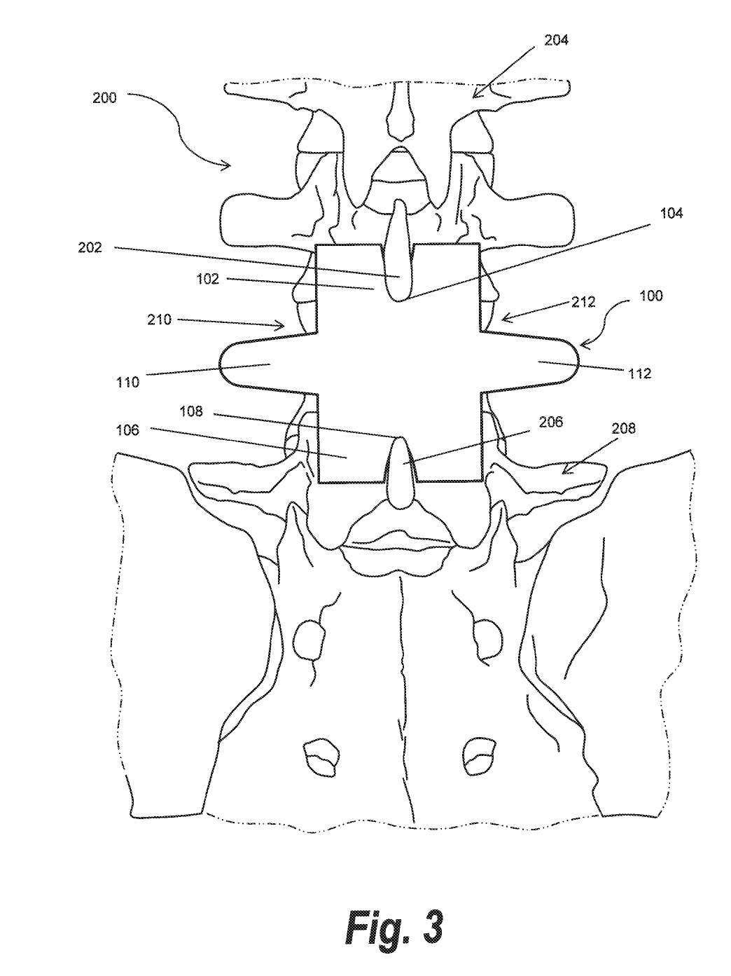

FIG. 3 illustrates the compressible and expandable spinal graft 100 after placement on or within the spine 200. The notch 104 of the superior arm 102 is positioned to receive and contact against a superior spinous process 202 of a superior vertebra 204. The notch 108 of the inferior arm 106 is positioned to receive and contact against an inferior spinous process 206 of an inferior vertebra 208. The first wing 110 extends into a first vertebral gutter 210. The second wing 112 extends into a second vertebral gutter 212. The location of the spinal graft 100 in the spine 200 is for illustration purposes only as the spinal graft 100 may span multiple vertebra and may be located in various regions of the spine 200.

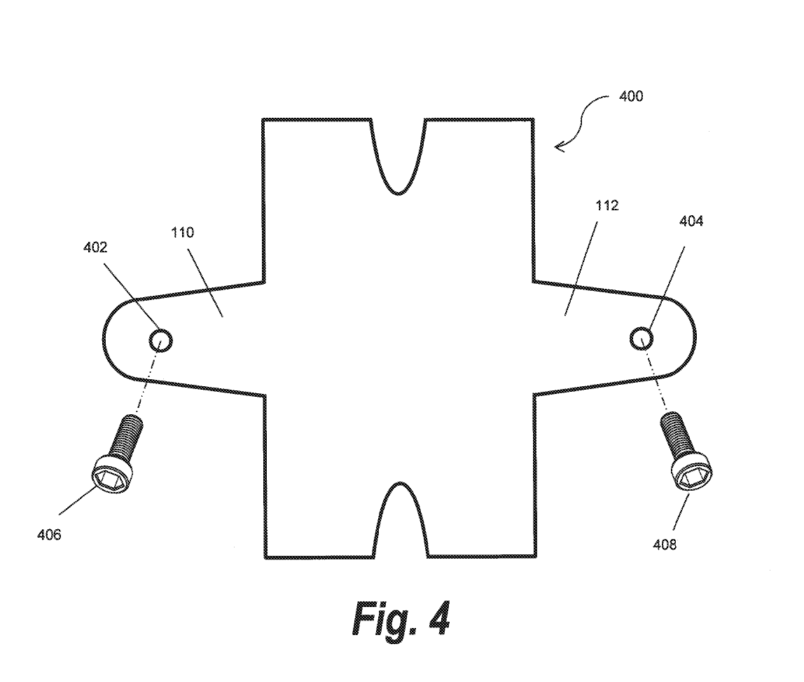

FIG. 4 illustrates an compressible and expandable graft 400 according to an alternative embodiment. As illustrated, the graft 400 includes openings (402, 404) centrally located in each wing (110, 112). Each opening (402, 404) is adapted to receive at least one securing device such as, for example, a screw (406, 408).

According to an alternative embodiment (not shown), a rod (or plate) is used to aid in prevention of movement. According to one embodiment, prevention of movement allows the bone graft to attach or fuse. According to such an embodiment, screws are placed above and below any bone, such as vertebrae, fused or stabilized by the graft. The screws and rods can be optionally removed at a later time.

The compressible and expandable grafts as provided here may be made of one or more materials suitable for implantation into the spine of a mammalian patient such as, for example, a human. Materials may be biocompatible with a mammalian patient and/or may have one or more surface coatings or treatments that enhance biocompatible and also reduce or prevent adhesion, nerve damage, pain, and graft migration. Such graft materials may include one or more materials having sufficient load capability and/or strength to maintain the desired spacing between spinous processes and provide the desired stability.

According to one embodiment, the grafts as provided herein may optionally include one or more of cancellous bone, demineralized cancellous bone, allograft (fresh or fresh-frozen), freeze dried bone allograft, demineralized freeze dried bone allograft, cortical cancellous bone, or a combination thereof. According to one embodiment, any donors of bone are subject to proper screening. According to one embodiment, the grafts as provided herein may optionally include demineralized bone matrix (DBM) that has undergone a process whereby the mineral content is removed. According to one embodiment, demineralized bone matrix functions as a bone graft extender. According to one embodiment, demineralized bone matrix may be mixed with autograft bone or other bone provided herein. According to one embodiment, the autograft includes bone cells, proteins, and calcified matrix. The autograft bone may be harvested from any appropriate portion of a donor's body including, but not limited, a donor's iliac crests, rib or spine. According to one embodiment, demineralized bone matrix may be mixed with allograft bone or other bone provided herein. Any allograft donor bone material may be obtained from a tissue bank. According to one embodiment, the allograft donor bone materials is prepared for use by freezing or freeze-drying to limit rejection.

According to one embodiment, the grafts as provided herein may optionally include one or more natural or recombinant bone morphogenetic proteins (BMPs) that are used to stimulate new bone growth. According to a particular embodiment, the bone morphogenetic protein is BMP-2 and BMP-7. According to one embodiment, the grafts as provided herein that include one or more bone morphogenetic proteins (BMPs) are suitable for spinal fusion such as an anterior lumbar interbody fusion. According to one embodiment, the grafts as provided herein that include one or more bone morphogenetic proteins (BMPs) are suitable for anterior cervical fusions.

According to one embodiment, the grafts as provided herein may optionally include one or more synthetic-based bone graft extenders such as, for example, calcium phosphate, tricalcium phosphate, phosphate, calcium sulfate, and bioactive glass. According to one embodiment, the synthetic-based bone graft extender is osteoconductive and biodegradable. The one or more synthetic-based bone graft extenders aid in fusion without a risk for disease transfer. According to one embodiment, the grafts as provided herein may optionally include bone marrow aspirate. According to one embodiment, the grafts as provided herein may optionally include growth factors, such as platelet derived growth factor and TGF-β to enhance bone healing by promoting mesenchymal stem cell and osteoblast proliferation.

According to a particular embodiment, the graft is made from demineralized cancellous bone as set forth in U.S. Pub. No. 20120259425, the content of which is incorporated herein by reference in its entirety. According to one embodiment, the donated bone material is harvested, tested and sterilized by an accredited tissue bank.

According to one embodiment, the grafts as provided herein may optionally include one or more bioactive agents. Suitable bioactive agents include, but are not limited to, antimicrobials, antibiotics, antimyobacterial, antifungals, antivirals, antineoplastic agents, antitumor agents, agents affecting the immune response, blood calcium regulators, agents useful in glucose regulation, anticoagulants, antithrombotics, antihyperlipidemic agents, cardiac drugs, thyromimetic and antithyroid drugs, adrenergics, antihypertensive agents, cholnergics, anticholinergics, antispasmodics, antiulcer agents, skeletal and smooth muscle relaxants, prostaglandins, general inhibitors of the allergic response, antihistamines, local anesthetics, analgesics, narcotic antagonists, antitussives, sedative-hypnotic agents, anticonvulsants, antipsychotics, anti-anxiety agents, antidepressant agents, anorexigenics, non-steroidal anti-inflammatory agents, steroidal anti-inflammatory agents, antioxidants, vaso-active agents, bone-active agents, osteogenic factors, antiarthritics, diagnostic agents and progenitor cells, or any combination thereof.

According to one embodiment, that grafts as provided herein include one or more synthetic polymers. According to one embodiment, the synthetic polymer is a polycaprolactone, collagen and open porosity polylactic acid polymer. According to one embodiment, the synthetic polymer is a hydrated polymer or hydrogel.

According to one embodiment, the grafts as provided herein are compressible and, thus, the grafts can be flattened or narrowed under pressure. According to one embodiment, the grafts as provided herein are expandable and, thus, the grafts can increase in size, volume or otherwise become enlarged. According to one embodiment, the grafts as provided herein are compressible and expandable as result of introduction of at least one flowable composition to one or more of the graft components. According to one embodiment, the at least one flowable composition includes at least one liquid that is introduced to the graft components while in powder form thus forming a solid. The at least one liquid includes, but is not limited to, of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, PRP, a flowable birth tissue composition, or a combination thereof.

The compressible and expandable grafts as provided herein may be coated, treated, or otherwise include an effective amount of a flowable composition including a birth tissue composition. According to one embodiment, the birth tissue composition includes one or more placental tissue components. The birth tissue composition may be formulated as a resorbable adhesion barrier allograft that is applied to the outer surface of the graft. According to an alternative embodiment, the birth tissue composition may be formulated as an injectable formulation or a flowable formulation that is introduced directly onto or into the grafts (i.e., mixed). According to either embodiment, placental tissue components and amniotic fluid must first be obtained from a seronegative, healthy mammal. Potential birth tissue donors providing informed consent are pre-screened during an examination of pre-natal medical records and blood test results. A comprehensive medical history and behavior risk assessment is obtained from the donor prior to donation incorporating U.S. Public Health Service guidelines. Discussions with the physician(s), veterinarian, and/or the donor mother are conducted to identify circumstances that may lead to the exclusion of the donor or donated tissue. Additionally, a physical exam is performed on the donor to determine whether there is evidence of high risk behavior or infection and to determine the overall general health of the donor.

Infectious disease testing of donor blood specimens is performed for each tissue donor on a specimen collected at the time of donation or within seven days prior to or after donation. Advantageously, the methods that are used to screen for a communicable disease follow the regulations as set forth by the Federal Drug Administration and the American Association of Tissue Banks. Exemplary infectious disease testing includes, but is not limited to, antibodies to the human immunodeficiency virus, type 1 and type 2 (anti-HIV-1 and anti-HIV-2); nucleic acid test (NAT) for HIV-1; hepatitis B surface antigen (HBsAg); total antibodies to hepatitis B core antigen (anti-HBc—total, meaning IgG and IgM); antibodies to the hepatitis C virus (anti-HCV); NAT for HCV; antibodies to human T-lymphotropic virus type I and type II (anti-HTLV-I and anti-HTLV-II); and syphilis (a non-treponemal or treponemal-specific assay may be performed).

Birth tissue is preferably recovered from a full-term Cesarean delivery of a newborn. Alternatively, birth tissue is recovered from a full-term vaginal delivery of a newborn. The subsequent steps of preparing the birth tissue material are performed in a controlled environment (i.e., certified biological safety cabinet, hood or clean room). Instruments, solutions, and supplies coming into contact with the birth tissue material during processing are sterile. All surfaces coming in contact with the birth tissue material intended for transplant are either sterile or draped using aseptic technique.

Once recovered, one or more of the placental tissue components can be removed via a sterile saline solution rinse, blunt dissection, scalpel, or a combination thereof, if necessary. According to one embodiment, umbilical cord, chorionic membrane, and other gelatins, fluids, cells and extracellular matrix are removed and discarded, leaving the amniotic membrane for further processing. Preferably, the birth tissue material is subject to preparation no more than four hours after recovery to preserve cell viability.

The retained placental tissue components can be placed in a sterile transport solution after aseptic recovery. The sterile transport solution is used to provide an advantageous medium to the natural function of the placental tissue components prior to processing. For example, calcium-rich water can be used as the sterile transport solution to provide a medium to drive undifferentiated cells to become osteogenic when implanted. Throughout the preparation of the birth tissue material, various methods can be used to drive undifferentiated cells to differentiate into specialized cell types including, but not limited to, transport solutions, soaks, particular temperature ranges, and hyperbaric pressure.

The sterile transport solution may include sodium chloride (NaCl) in a concentration range from typically about 0.1% to typically about 35% by weight. The sterile transport solution can also include one or more of Minimum Essential Medium, Dulbecco's Modified Eagle's Medium, Plasma Lyte-A, human albumin 25% solution, calcium-rich water, alkaline ionized water, or acidic ionized water. After delivery to the processing facility, the weight of the placental tissue components can be determined. Thereafter, the placental tissue components can be transferred aseptically to a sterile dish containing Plasma Lyte-A and stored in a quarantine refrigerator pending further processing. The placental tissue components can be removed from the Plasma Lyte-A and cryopreserved according to methods commonly used in the art. According to one embodiment, the cryopreserved components may then be morselized and formulated into an injectable form and/or a flowable material.

The birth tissue material compositions as described herein can be optionally mixed with or administered in combination with bioactive agents such as inflammatory inhibitors, antibiotics, cytokines, minerals, growth factors (e.g., fibrin and/or thrombin), wound healing agents, hyaluronic acid, cellular attractant and scaffolding reagents (e.g., fibronectin) antibiotics, chemotherapeutic agents, antigens, antibodies, enzymes, NSAIDs, muscle relaxants, vectors for gene delivery and hormones.

The grafts as provided herein may be used in a variety of surgeries that require bone grafts. According to one embodiment, the grafts as provided herein may be used in arthrodesis surgery for linking, fusing or otherwise welding bones together. Such a surgery may function to prevent motion between vertebral bodies, repair a fracture or stabilize a spinal deformity.

The grafts as provided herein may be used in decompression surgery. Decompression surgery and subsequent placement of the compressible and expandable grafts as provided here are typically performed by an orthopedic surgeon. Surgery is initiated by making a skin incision down the middle of the back over the appropriate vertebrae. The length of the incision depends on the number of laminae that will be subject to the decompression surgery procedure chosen. The strong back muscles are then split and moved to either side of each lamina exposing each vertebra. During a laminectomy, the specific lamina and ligamentum flavum are then removed. Optionally, the surgeon may then retract the dural sac and nerve root to remove any bone spurs or thickened ligaments. The facet joints may then be undercut or trimmed. The spinal fusion to stabilize the spine may then be performed.

According to one embodiment, the spinal fusion or spinal stabilization process may be carried out by placement of an compressible and expandable graft as provided herein. Such a process includes the step of positioning an compressible and expandable graft between the spinal processes of the superior and inferior vertebra which are above and below the lamina or laminae subject to decompression surgery. The compressible and expandable grafts as provided herein may be adapted to be inserted between spinous processes at any region in the spine. Although typically grafts may be inserted in the lumbar region (e.g., between L3 and L5), the compressible and expandable grafts as provided herein may be positioned into other regions such as for example, the thoracic or cervical region. The compressible and expandable grafts as provided herein may also span multiple vertebra as in the case of removal or alteration of multiple laminae during a laminectomoy or laminotomy. According to one embodiment, the graft includes cortical bone on one side and cancellous bone on an opposite side. According to one embodiment, the graft as provided herein is shaped to fit between the facets and pedicles in the spine.

A method of delivering at least one flowable composition to a spinal region is provided. The method includes the step of introducing at least one flowable composition to a graft as provided herein that includes a shell having a first side and a second opposing side, the first side comprising cortical bone, and the second, opposing side comprising compressed cancellous bone. The graft is shaped to be placed between facets and pedicals in a spine. According to a particular embodiment, the graft includes at least one material such as, for example, cancellous bone, demineralized cancellous bone, fresh allograft, frozen allograft freeze dried bone allograft, demineralized freeze dried bone allograft, cortical cancellous bone, or a combination thereof to form the graft. The method further includes the step of introducing the graft to the spinal region of a patient in need treatment. According to one embodiment, the at least one liquid component is delivered in a time release manner. According to one embodiment, the flowable composition is delivered over a period of time from about 1 minutes to about 1 week. According to such an embodiment, the resulting graft acts as a vector for delivery of the flowable composition. According to one embodiment, the at least flowable composition includes at least one liquid component that is a biologic such as, for example, one or more vitamins (calcium, magnesium, vitamin D3, vitamin K, vitamin C, collagen, glucosamine, chondroitin, vitamin B12, iron, copper, zinc, boron, manganese), nutrients, inflammatory inhibitors, antibiotics, cytokines, minerals, growth factors (e.g., fibrin and/or thrombin), wound healing agents, hyaluronic acid, cellular attractant and scaffolding reagents (e.g., fibronectin) antibiotics, chemotherapeutic agents, antigens, antibodies, enzymes, NSAIDs, muscle relaxants, Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, PRP, a flowable human birth tissue composition, or any combination thereof. According to one embodiment, the biologic aids in fusing the spinal region. According to one embodiment, the biologic aids in the healing cascade.

According to the graft embodiment as illustrated in the figures provided herein, the superior and inferior notches are engaged with the superior and inferior lamina of the superior and inferior vertebra, in no specific order, such that the space between the superior and inferior vertebra is maintained and the spine is stabilized (see e.g., FIG. 3). The first and second wings of the graft are simultaneously positioned within the vertebral gutters. According to a preferred embodiment, the graft remains in place between the laminae without the need for introducing any securing hardware such as screws, plates, or rods. Alternatively, the graft may be secured by introduction of screws through the first and second wings and into the vertebral gutters (see e.g., FIG. 4). According to such an embodiment, other stabilizing hardware such as, for example, plates or rods may optionally be used in conjunction with the screws.

A kit is also provided that includes one or more grafts as provided herein. The kit may include various sizes of compressible and expandable grafts depending on the location of where the graft may be utilized in the spine. The kit may further include tools or other devices useful in selecting, inserting, positioning, and/or securing one or more grafts. Tools and devices may include, for example, one or more pins, screws, rods, plates, wires, cables, straps, surgical rope, sutures, or other devices typically used for positioning and securing the grafts. The kit further includes at least one set of instructions.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

We claim:

1. A graft comprising
a shell having a first side and a second opposing side,
wherein the first side comprises cortical bone,
wherein the second, opposing side comprises compressed cancellous bone,
wherein the graft is shaped to be placed between facets and pedicals in a spine,
wherein the compressed cancellous bone is infused with and delivers at least one flowable composition to a spinal region.

2. The graft of claim 1, wherein the flowable composition includes at least one liquid component selected from the group consisting of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, PRP, and a combination thereof.

3. The graft of claim 2, wherein the flowable composition includes a flowable birth tissue material composition.

4. The graft of claim 4, wherein the flowable birth tissue composition comprises one or more components of the placental organ.

5. The graft of claim 4, wherein the one or more of the components of the placental organ is selected from the group consisting of umbilical cord, umbilical cord blood, chorionic membrane, amniotic membrane, amnion membrane, Wharton's jelly, amniotic fluid, and other placental gelatins, cells, and extracellular material.

6. The graft of claim 1, further comprising one or more natural or recombinant bone morphogenetic proteins (BMPs).

7. The graft of claim 6, wherein the bone morphogenetic protein is BMP-2, BMP-7 or a combination thereof.

8. The graft of claim 1, further comprising one or more synthetic-based bone graft extenders.

9. The graft of claim 8, wherein the one or more synthetic-based bone graft extenders is calcium phosphate, tricalcium phosphate, phosphate, calcium sulfate, bioactive glass, or a combination thereof.

10. The graft of claim 1, further comprising one or more bioactive agents.

11. The graft of claim 1, further comprising one or more synthetic polymers.

12. A method of stabilizing a vertebral region of a spine comprising
providing the graft of claim 1,
applying the graft to a vertebral region of the spine such that the second, opposing side is placed into a spinal gutter.

13. The method of claim 12, further comprising the step of securing the graft to the vertebral region with at least one screw, rod, plate, or a combination thereof.

14. A method for delivering at least one flowable composition to a spinal region comprising
introducing at least one flowable composition to the second, opposing side of the graft of claim 1; and
introducing the graft to the spinal region of a patient in need treatment,
wherein at least one flowable composition is delivered in a time release manner.

15. The graft of claim 14, wherein the flowable composition includes at least one liquid component selected from the group consisting of Minimum Essential Medium, Dulbecco's Modified Eagle's Medium, Plasma Lyte-A, human albumin 25% solution, calcium-rich water, alkaline ionized water, acidic ionized water, pharmaceutical grade water, neutral pH saline, blood, PRP, and a combination thereof.

16. The graft of claim 14, wherein the flowable composition includes a flowable birth tissue material composition.

17. A kit comprising
a graft as provided in claim 1,
optionally, at least one screw, rod, or combination thereof for securing the graft to the spine; and
instructions for use thereof.
